(19) United States
(12) Patent Application Publication
(10) Pub. No.: US 2024/0341695 A1
(43) Pub. Date: Oct. 17, 2024

(54) PREDICTING CLASSIFICATION LABELS FOR BIOELECTRIC SIGNALS USING A NEURAL NETWORK

(71) Applicant: Tiposi, Inc., Milpitas, CA (US)

(72) Inventors: Fuhao Li, Milpitas, CA (US); Lijun Zhang, Milpitas, CA (US); Shih Hsiung Mo, Milpitas, CA (US)

(73) Assignee: Tiposi, Inc., Milpitas, CA (US)

(21) Appl. No.: 18/609,211

(22) Filed: Mar. 19, 2024

Related U.S. Application Data
(60) Provisional application No. 63/493,117, filed on Mar. 30, 2023.

(51) Int. Cl. A61B 5/00; A61B 5/0507

(52) U.S. Cl. CPC A61B 5/7267; A61B 5/0042; A61B 5/0507; A61B 5/4064; A61B 5/6803; A61B 5/7257; A61B 5/7278; A61B 5/7282; A61B 5/742; A61B 2562/0228

(57) ABSTRACT

Embodiments of a system for training a classification neural network are provided. The system is configured to receive a first set of simulated bioelectric signals and patient bioelectric signals from a first computing device and a second set of simulated bioelectric signals from a second computing device, generate a compensation factor for the second computing device based on the first set of simulated bioelectric signals and the second set of simulated bioelectric signals, generate compensated patient bioelectric signals based on the compensation factor and the patient bioelectric signals, and train the classification neural network based on the compensated patient bioelectric signals, the second set of simulated bioelectric signals and the compensation factor. The classification neural network is trained to predict a classification label for each of one or more bioelectric signals.

### TECHNOLOGICAL FIELD

The present disclosure generally relates to imaging-based health monitoring apparatuses, and more particularly relates to systems and methods for classifying bioelectric signals using a neural network.

### BACKGROUND

Stroke is a critical medical condition that is characterized by sudden disruption or interruption of blood flow to the brain of a patient. The stroke may result in severe neurological impairment or even fatality if not promptly diagnosed and treated.

Typically, detection of stroke conditions in a human body relies on clinical assessment, which may be subjective and time-consuming. In certain cases, imaging techniques such as Magnetic Resonance Imaging (MRI) and Computed Tomography (CT) scans are utilized for detecting stroke conditions. However, these methods may be expensive, resource-intensive, rely on user's knowledge, and may not always be readily accessible, especially in remote or underserved areas.

### BRIEF SUMMARY

The present disclosure may provide a system, a method and a computer program product that enables automated determination of a heath condition for a patient, particularly to detect stroke conditions for the patient.

In an aspect, a system for training a classification neural network for deployment on a second computing device is disclosed. The system comprises a memory configured to store a classification neural network and computer-executable instructions. The system comprises one or more processors operably connected to the memory and configured to execute the computer-executable instructions to receive a first set of simulated bioelectric signals and patient bioelectric signals from a first computing device and a second set of simulated bioelectric signals from a second computing device. The one or more processors are further configured to generate a compensation factor for the second computing device based on the first set of simulated bioelectric signals and the second set of simulated bioelectric signals. The one or more processors are further configured to generate compensated patient bioelectric signals based on the compensation factor and the patient bioelectric signals. The one or more processors are further configured to train the classification neural network based on the compensated patient bioelectric signals, the second set of simulated bioelectric signals and the compensation factor. In an example, the classification neural network is trained to predict a classification label for each of one or more bioelectric signals.

In another aspect, a system for classifying patient bioelectric data is disclosed. The system comprises a memory configured to store a trained classification neural network and computer-executable instructions, and one or more processors operably connected to the memory. The one or more processors are configured to execute the computer-executable instructions to receive patient bioelectric data relating to an anatomical part of a patient. The one or more processors are configured to classify the patient bioelectric data using a trained classification neural network to associate at least one classification label with the patient bioelectric data. The classification neural network is trained based on patient bioelectric signals collected by a first computing device and compensated based on a compensation factor for a second computing device. The compensation factor is determined based on a first set of simulated bioelectric signals collected by the first computing device and a second set of simulated bioelectric signals collected by the second computing device. Moreover, the classification label indicates one of: a presence, or an absence of at least one health condition, associated with the anatomical part. Further, the one or more processors are configured to output the patient bioelectric data with the corresponding at least one classification label.

In yet another aspect, a method for predicting classification labels for biological signals is disclosed. The method comprises receiving a first set of simulated bioelectric signals and patient bioelectric signals from a first computing device and a second set of simulated bioelectric signals from a second computing device. The method further comprises generating a compensation factor for the second computing device based on the first set of simulated bioelectric signals and the second set of simulated bioelectric signals, and generating compensated patient bioelectric signals based on the compensation factor and the patient bioelectric signals. The method further comprises training a classification neural network based on the compensated patient bioelectric signals, the second set of simulated bioelectric signals and the compensation factor. The classification neural network is trained to predict a classification label for each of one or more bioelectric signals.

In yet another aspect, a computer program product for training a classification neural network for predicting classification labels for biological signals is disclosed. The computer program product comprises a non-transitory computer readable medium having stored thereon computer executable instructions, which when executed by one or more processors, cause the one or more processors to carry out operations. The operations comprise receiving a first set of simulated bioelectric signals and patient bioelectric signals from a first computing device and a second set of simulated bioelectric signals from a second computing device. The operations further comprise generating a compensation factor for the second computing device based on the first set of simulated bioelectric signals and the second set of simulated bioelectric signals, and generating compensated patient bioelectric signals based on the compensation factor and the patient bioelectric signals. The operations further comprise training a classification neural network based on the compensated patient bioelectric signals, the second set of simulated bioelectric signals and the compensation factor. The classification neural network is trained to predict a classification label for each of one or more bioelectric signals.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

Having thus described example embodiments of the disclosure in general terms, reference will now be made to the accompanying drawings, which are not necessarily drawn to scale, and wherein:

FIG. 1 illustrates a block diagram of a network environment comprising a system for training a classification neural network, in accordance with one or more embodiments of the present disclosure;

FIG. 2 illustrates an exemplary block diagram of the system for training the classification neural network, in accordance with an example embodiment of the present disclosure;

FIG. 3 illustrates a reference artificial brain model, in accordance with an example embodiment of the present disclosure;

FIG. 4 illustrates a flowchart of a method for pre-processing measured bioelectric signals, in accordance with an example embodiment of the present disclosure;

FIG. 5A illustrates a flowchart of a training process of the classification neural network, in accordance with one or more example embodiments;

FIG. 5B illustrates an exemplary block diagram of a training process of the classification neural network, in accordance with different embodiments of the present disclosure;

FIG. 5C illustrates a block diagram for training the classification neural network, in accordance with an example embodiment of the present disclosure;

FIG. 6 illustrates a flowchart of a method for implementing the classification neural network, in accordance with an example embodiment of the present disclosure;

FIG. 7 illustrates a schematic diagram of an architecture of the classification neural network, in accordance with an example embodiment of the present disclosure;

FIG. 8A illustrates an example schematic diagram of a re-training process of the classification neural network, in accordance with an example embodiment of the present disclosure; and

FIG. 8B illustrates an example flowchart of the re-training process of the classification neural network, in accordance with an example embodiment of the present disclosure.

### DETAILED DESCRIPTION

In the following description, for purposes of explanation, numerous specific details are set forth in order to provide a thorough understanding of the present disclosure. It will be apparent, however, to one skilled in the art that the present disclosure may be practiced without these specific details. In other instances, systems and methods are shown in block diagram form only in order to avoid obscuring the present disclosure.

Reference in this specification to “one embodiment” or “an embodiment” means that a particular feature, structure, or characteristic described in connection with the embodiment is included in at least one embodiment of the present disclosure. The appearance of the phrase “in one embodiment” in various places in the specification are not necessarily all referring to the same embodiment, nor are separate or alternative embodiments mutually exclusive of other embodiments. Further, the terms “a” and “an” herein do not denote a limitation of quantity, but rather denote the presence of at least one of the referenced items. Moreover, various features are described which may be exhibited by some embodiments and not by others. Similarly, various requirements are described which may be requirements for some embodiments but not for other embodiments.

Some embodiments of the present disclosure will now be described more fully hereinafter with reference to the accompanying drawings, in which some, but not all, embodiments of the disclosure are shown. Indeed, various embodiments of the disclosure may be embodied in many different forms and should not be construed as limited to the embodiments set forth herein; rather, these embodiments are provided so that this disclosure will satisfy applicable legal requirements. Like reference numerals refer to like elements throughout. Also, reference in this specification to “one embodiment” or “an embodiment” means that a particular feature, structure, or characteristic described in connection with the embodiment is included in at least one embodiment of the present disclosure. The appearance of the phrase “in one embodiment” in various places in the specification are not necessarily all referring to the same embodiment, nor are separate or alternative embodiments mutually exclusive of other embodiments. Further, the terms “a” and “an” herein do not denote a limitation of quantity, but rather denote the presence of at least one of the referenced item. Moreover, various features are described which may be exhibited by some embodiments and not by others. Similarly, various requirements are described which may be requirements for some embodiments but not for other embodiments.

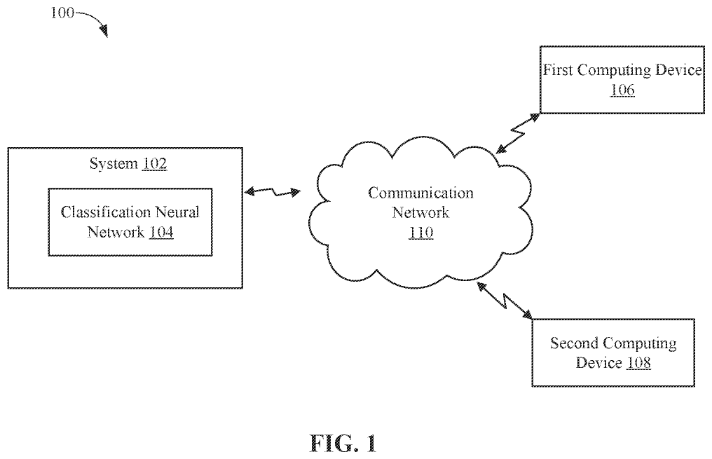

The embodiments are described herein for illustrative purposes and are subject to many variations. It is understood that various omissions and substitutions of equivalents are contemplated as circumstances may suggest or render expedient but are intended to cover the application or implementation without departing from the spirit or the scope of the present disclosure. Further, it is to be understood that the phraseology and terminology employed herein are for the purpose of the description and should not be regarded as limiting. Any heading utilized within this description is for convenience only and has no legal or limiting effect. Turning now to FIG. 1-FIG. 8, a brief description concerning the various components of the present disclosure will now be briefly discussed. Reference will be made to the figures showing various embodiments of a system for providing a user with an interactive map.

Embodiments of present disclosure provide techniques for training a classification neural network such that the classification neural network can be implemented on various health monitoring devices. In an example, the health monitoring devices may be Microwave imaging (MWI) based devices that uses microwave signals to image an anatomical part or a body part of a patient. Herein, the classification neural network uses deep learning techniques to classify bioelectric signals sensed by various microwave imaging devices to detect any anomaly in the body part of the patient. For example, bioelectric signals may correspond to brain waves of a patient. In such a case, the classification neural network is trained to accurately detect if stroke condition or stroke symptoms are present within the brain waves of the patient.

To this end, the classification neural network is used to classify the bioelectric signals to ensure accuracy in classification. Embodiments of the present disclosure provide techniques to improve the accuracy in classifying bioelectric signals when the classification neural network is deployed on a new device, i.e., a second computing device.

It may be noted, early detection and diagnosis of an anomaly or a health condition, such as stroke, is crucial for effective treatment and to prevent long-term disabilities. In certain cases, imaging-based techniques, such as computed tomography (CT) and magnetic resonance imaging (MRI) are used for stroke detection. However, these techniques may have high cost, may expose a patient to ionizing radiation, may be time and resource intensive, and may require expert analysis.

Recently, microwave-based imaging (MWI) techniques are being used for non-invasive, low-cost, and real-time imaging of an anatomical part or a body part of the patient. In particular, microwave signals are used to produce images of the anatomical part, such as the brain, and the image may be used to identify areas of abnormality, such as abnormal blood flow within the brain. Using the microwave signals indicating an image of the brain, medical professionals are able to diagnose strokes. However, analyzing patient outcomes manually may be time consuming, costly, and susceptible to human judgements, errors, and bias.

Further, deep learning-based methods are used with imaging-based devices for anomaly detection in patients. For example, the anomaly may be related to brain stroke. The deep learning-based methods may enable fast and accurate detection of stroke based on the collected microwave signals by an imaging device. However, the microwave signals collected by different devices are inconsistent from each other.

Typically, a scanning device (referred to as, a first computing device) may be used to scan a brain and collect data. For example, the first computing device may be in the form of a helmet. Further, a trained deep-learning model may be deployed on the first computing device to analyze and classify the collected data. For example, the deep-learning model may be trained over time based on, first, a large amount of simulation data collected by scanning artificial heads to verify algorithms of the deep-learning model, and second, real brain data. In particular, the first computing device may be used to collect a large amount of data from real heads, i.e., real brains of patients. The data collected from the real heads may include data relating to normal patients or normal brains and patients with abnormal or stroke conditions. Further, the deep-learning model is trained over time based on the collected data from real patients and user feedback so that accuracy of the deep-learning model in predicting stroke in a brain is very high, for example, 99%. After completing the training and validation of the deep-learning model, the deep learning model may have to be deployed on other computing devices for commercialization.

To this end, some embodiments of the present disclosure are based on a realization that when the deep learning model trained on the data collected by the first computing device is deployed on another scanning device (referred to as, a second computing device), an accuracy rate of the trained deep learning model is very low for data collected by the second computing device.

Some embodiments are based on a realization that the second computing device cannot use the deep learning model of the first computing device directly as there exists certain hardware differences between the different computing devices. These differences may arise due to, for example, manufacturing of antennas or sensors, manufacturing of circuit setup, operating environmental condition, data processing parameters, and errors in circuit components. Therefore, the patient data collected by the first computing device from the real patients cannot be used directly for training the deep learning model to be implemented on the second computing device. For example, direct deployment of the deep learning model across different devices may cause a significant decrease in performance of the deep learning model for classifying the patient data to detect stroke.

Some embodiments are based on a realization that collecting patient data from real patients using the second computing device (or every new computing device on which the deep learning model is to be deployed) is time-consuming, resource intensive and logistically challenging.

Some embodiments are based on a realization that the second computing device may be used on artificial heads to collect simulation data. Subsequently, the deep learning model may get trained on the simulation data collected by the second computing device. However, the training of the deep learning model only on the simulation data does not yield good outcomes or high accuracy for the second computing device.

In some cases, direct deployment of the deep learning model onto the second computing device may result in complete loss of model's ability to classify signals collected by the second computing device. Therefore, there is a need to address inconsistency in data collected by different devices before using collected data for training the deep learning model for deployment on the different devices to improve the accuracy of the deep learning model.

Embodiments of the present disclosure provide systems and methods to overcome inconsistency in bioelectric signals collected from different devices to ensure accurate training of the deep learning model. As a result, accuracy of deep learning model (referred to as a classification neural network) is improved, specifically, when the model is deployed on a new device.

FIG. 1 illustrates a block diagram of a network environment 100 comprising a system 102 implemented to train a classification neural network 104, in accordance with one or more example embodiments of the present disclosure. In an example, the classification neural network 104 is trained in a manner such that inaccuracies due to inconsistent data collected by different devices are eliminated.

In this regard, the system 102 is coupled to a first computing device 106 and a second computing device 108 via a communication network 110. For example, the first computing device 106 is an old or an existing computing device having enough device-specific data for training. In an example, the first computing device 106 is the first device on which the classification neural network 104 is deployed. Subsequently, artificial, or real bioelectric signals collected by the first computing device 106 are used to train the classification neural network 104. Further, the second computing device 108 is a new computing device that does not have enough device-specific data for training. Additional, fewer, or different components may be provided.

The above presented components of the system 102 can be implemented in hardware, firmware, software, or a combination thereof. Though depicted as a separate entity in FIG. 1, it is contemplated that the system 102 may be implemented as a module of any of the first computing device 106 and the second computing device 108.

The communication network 110 may be wired, wireless, or any combination of wired and wireless communication networks, such as cellular, Wi-Fi, internet, local area networks, or the like. In some embodiments, the communication network 110 may include one or more networks such as a data network, a wireless network, a telephony network, or any combination thereof. It is contemplated that the data network may be any local area network (LAN), metropolitan area network (MAN), wide area network (WAN), a public data network (e.g., the Internet), short range wireless network, or any other suitable packet-switched network, such as a commercially owned, proprietary packet-switched network, e.g., a proprietary cable or fiber-optic network, and the like, or any combination thereof. In addition, the wireless network may be, for example, a cellular network and may employ various technologies including enhanced data rates for global evolution (EDGE), general packet radio service (GPRS), global system for mobile communications (GSM), Internet protocol multimedia subsystem (IMS), universal mobile telecommunications system (UMTS), etc., as well as any other suitable wireless medium, e.g., worldwide interoperability for microwave access (WiMAX), Long Term Evolution (LTE) networks (for e.g. LTE-Advanced Pro), 5G New Radio networks, ITU-IMT 2020 networks, code division multiple access (CDMA), wideband code division multiple access (WCDMA), wireless fidelity (Wi-Fi), wireless LAN (WLAN), Bluetooth, Internet Protocol (IP) data casting, satellite, mobile ad-hoc network (MANET), and the like, or any combination thereof.

In an example, the classification neural network 104 is a deep-learning model or a deep-learning neural network. The classification neural network 104 is used for feature categorization, and only allows one output response for every input pattern. For example, a classification category that has a highest probability value is chosen by the classification neural network 104. The classification neural network 104 may be integrated with predictive neural networks in a hybrid system for classifying bioelectric signals and predicting presence of an anomaly, such as stroke in a patient. In this regard, the classification neural network 104 may extract features of microwave signals relating to an anatomical part, such as brain. Further, the classification neural network 104 may learn patterns and features of normal condition as well as anomalies, such as stroke, within the features of the images. Based on the learnt patterns and features, the classification neural network 104 may classify microwave signals for different patients or different parts of the anatomical part based on one or more category labels. In an example, the classification neural network 104 includes a plurality of one-dimensional (1D) convolutional neural networks (CNNs).

In an example, at first, the classification neural network 104 may be deployed on the first computing device 106. For example, the first computing device 106 may be a microwave-imaging device that includes an antenna array. The first computing device 106 is configured to transmit microwave signals and measure reflected microwave signals from an object. Pursuant to present example, the object is a human body, i.e., an anatomical part of the body of a patient. To this end, the first computing device 106 may measure or collect large amounts of data from both real human anatomical part as well as simulated or artificial anatomical part. In accordance with an example, the anatomical part may be a brain. Subsequently, the first computing device 106 may measure or collect patient bioelectric signals that are measured from real human brain of patients, as well as a first set of simulated bioelectric signals that are measured from artificial or simulated brains.

It may be noted that the present disclosure describes collecting bioelectric signals from real or artificial brains, however, this should not be construed as a limitation. In other examples of the present disclosure, the bioelectric signals may be collected from other anatomical parts of the body, such as heart, kidney, lungs, etc.

When the classification neural network 104 is deployed on the first computing device 106, the classification neural network 104 gets trained based on the data collected by the first computing device 106. In particular, the classification neural network 104 gets trained on the patient bioelectric signals and the first set of simulated bioelectric signals.

Thereafter, the classification neural network 104 is deployed on the second computing device 108. In an example, the second computing device 108 is also a microwave-imaging device that includes an antenna array. The second computing device 108 is also configured to transmit microwave signals and measure reflected microwave signals from an object, i.e., the anatomical part or the brain. To this end, the second computing device 108 is a new device and may not have been used for measuring patient bioelectric signals. Typically, the classification neural network 104 may have to be calibrated before deploying it on the second computing device 108. In order to calibrate the classification neural network 104 for the second computing device 108, the newly produced second computing device 108 uses artificial brains to gather time-domain signal data, referred to as a second set of simulated bioelectric signals.

In operation, the system 102 is configured to receive the first set of simulated bioelectric signals and the patient bioelectric signals from the first computing device 106. The system 102 is also configured to receive the second set of simulated bioelectric signals from the second computing device 108. It may be noted that the first computing device 106 may collect or measure the first set of simulated bioelectric signals and the second computing device 108 may collect or measure the second set of simulated bioelectric signals using an artificial or a simulated brain. In an example, the artificial brain may be a software-simulated brain or a hardware reference brain model.

In an example, the hardware reference brain model may be a device, such as a head phantom. The head phantom may mimic human head variations, i.e., various signals or brain waves in brains. The head phantom may be manufactured using a jelly or a jelly-like material.

In an example, a same or similar head phantom(s) or artificially simulated brain model(s) may be used for collecting the first set of simulated bioelectric signals using the first computing device 106 and the second set of bioelectric signals using the second computing device 108. In another example, a same or similar intensity electric signals may be generated within different head phantoms or different artificially simulated brain models to enable the first computing device 106 and the second computing device 108 to measure the first set of simulated bioelectric signals and the second set of simulated bioelectric signals.

Based on the received first set of simulated bioelectric signals and the second set of bioelectric signals, the system 102 is configured to generate a compensation factor for the second computing device 108. In an example, the compensation factor may be generated based on a comparison of the first set of simulated bioelectric signals with the second set of simulated bioelectric signals. For example, a first signal from the first set of simulated bioelectric signals is compared with a second signal from the second set of simulated bioelectric signals such that the first signal and the second signal correspond to a same component in the head phantom(s). The components may be the same type of, for example, brain tissues, blood vessels, arteries, and veins, etc. For example, a difference between a parameter (such as intensity, photon energy, density, etc.) of the first signal and the second signal for the same component is determined. Such difference may be used to generate the compensation factor. For example, based on the comparison of each of the first set of simulated bioelectric signals of different types with corresponding type from the second set of simulated bioelectric signals, the compensation factor may be determined. In an example, the compensation factor may be a degree, a grade, a numerical value, etc.

Once the compensation factor is generated, the system 102 is configured to generate compensated patient bioelectric signals based on the compensation factor and the patient bioelectric signals collected by the first computing device 106. In an example, the patient bioelectric signals measured by the first computing device 106 is offset or adjusted based on the compensation factor. As the patient bioelectric signals are collected by the first computing device 106, compensating it based on the compensation factor makes it accurate and usable for the second computing device 108.

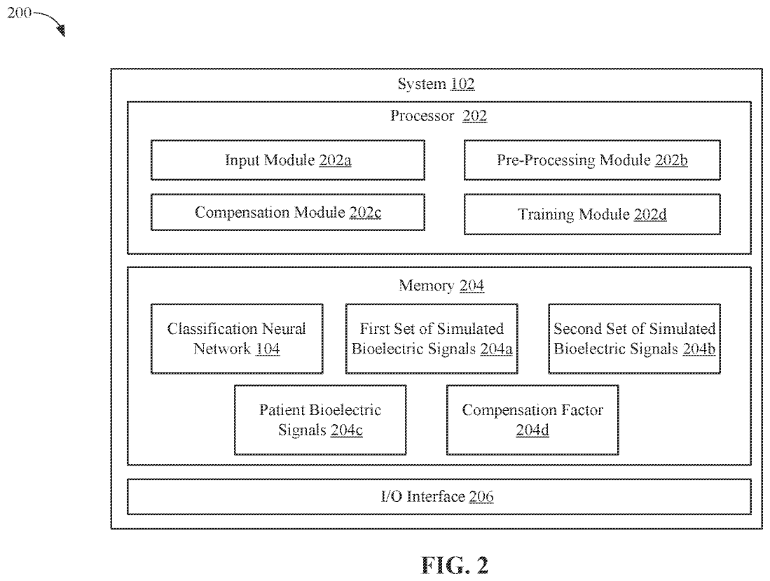

Thereafter, the system 102 is configured to train the classification neural network 104 based on the compensated patient bioelectric signals, the second set of simulated bioelectric signals and the compensation factor. In an example, the classification neural network 104 is trained to predict a classification label for each of one or more bioelectric signals. In particular, the classification neural network 104 is fed with bioelectric signals comprising the second set of simulated bioelectric signals, and the compensated patient bioelectric signals. For example, the second set of simulated bioelectric signals, and the compensated patient bioelectric signals may form training dataset for training the classification neural network 104. To this end, the compensated patient bioelectric signals closely match real data that would be collected by the second computing device 108. Therefore, the compensated patient bioelectric signals and the second set of simulated bioelectric signals are used to train the classification neural network 104. Once trained, the classification neural network 104 is deployed resulting in higher classification performance when deployed on the second computing device 108. Moreover, training the classification neural network 104 on the compensated patient bioelectric signals may reduce time and cost that would otherwise be required for developing and training a new model for the second computing device 108. Details of operations of the system 102 are described in conjunction with, for example, FIG. 2.

FIG. 2 illustrates an exemplary block diagram 200 of the system 102, in accordance with one or more example embodiments. FIG. 2 is explained in conjunction with FIG. 1.

The system 102 may include a processor 202, a memory 204, and an I/O interface 206. The processor 202 is configured to collect and/or analyze data from the memory 204, and/or any other data repositories available over the communication network 110 to compensate data for training of the classification neural network 104. Further, the processor 202 may include modules, depicted as, an input module 202a, a pre-processing module 202b, a compensation module 202c, and a training module 202d.

The I/O interface 206 may receive inputs and provide outputs for end user to view, such as render bioelectric signals, render classification labels, etc. In an example embodiment, the I/O interface 206 may present bioelectric signals measured by the second computing device 108 on a display, classification labels of the measured bioelectric signals, etc. It is further noted that the I/O interface 206 may operate over the communication network 110 to facilitate the exchange of information. As such, the I/O interface 206 may include a display and, in some embodiments, may also include a keyboard, a mouse, a joystick, a touch screen, touch areas, soft keys, one or more microphones, a plurality of speakers, or other input/output mechanisms. In one embodiment, the I/O interface 206 may comprise user interface circuitry configured to control at least some functions of one or more I/O interface elements such as a display and, in some embodiments, a plurality of speakers, a ringer, one or more microphones and/or the like.

In an example, the processor 202 may be embodied in a number of different ways. For example, the processor 202 may be embodied as one or more of various hardware processing means such as a coprocessor, a microprocessor, a controller, a digital signal processor (DSP), a processing element with or without an accompanying DSP, or various other processing circuitry including integrated circuits such as, for example, an ASIC (application specific integrated circuit), an FPGA (field programmable gate array), a microcontroller unit (MCU), a hardware accelerator, a special-purpose computer chip, or the like. As such, in some embodiments, the processor 202 may include one or more processing cores configured to perform independently. A multi-core processor may enable multiprocessing within a single physical package. Additionally, or alternatively, the processor 202 may include one or more processors configured in tandem via the bus to enable independent execution of instructions, pipelining and/or multithreading. Additionally, or alternatively, the processor 202 may include one or more processors capable of processing large volumes of workloads and operations to provide support for big data analysis. In an example embodiment, the processor 202 may be in communication with the memory 204 via a bus for passing information among components of the system 102.

In an example embodiment, the processor 202 is configured to train the classification neural network 104 and deploy the trained classification neural network 104 onto the second computing device 108 for collecting patient data. The classification neural network 104 may be trained based on compensated patient bioelectric signals, and second set of simulated bioelectric signals.

The memory 204 may be non-transitory and may include, for example, one or more volatile and/or non-volatile memories. In other words, for example, the memory 204 may be an electronic storage device (for example, a computer readable storage medium) comprising gates configured to store data (for example, bits) that may be retrievable by a machine (for example, a computing device like the processor 202). The memory 204 may be configured to store information, data, content, applications, instructions, or the like, for enabling the system 102 to carry out various functions in accordance with an example embodiment of the present disclosure. For example, the memory 204 may be configured to buffer input data for processing by the processor 202. As exemplarily illustrated in FIG. 2, the memory 204 may be configured to store instructions for execution by the processor 202. In some example embodiment, the memory 204 functions as a repository within the system. The memory 204 is configured to store the classification neural network 104.

As such, whether configured by hardware or software methods, or by a combination thereof, the processor 202 may represent an entity (for example, physically embodied in circuitry) capable of performing operations according to an embodiment of the present disclosure while configured accordingly. Thus, for example, when the processor 202 is embodied as an ASIC, FPGA or the like, the processor 202 may be specifically configured hardware for conducting the operations described herein.

Alternatively, as another example, when the processor 202 is embodied as an executor of software instructions, the instructions may specifically configure the processor 202 to perform the algorithms and/or operations described herein when the instructions are executed. However, in some cases, the processor 202 may be a processor specific device (for example, a mobile terminal or a fixed computing device) configured to employ an embodiment of the present disclosure by further configuration of the processor 202 by instructions for performing the algorithms and/or operations described herein. The processor 202 may include, among other things, a clock, an arithmetic logic unit (ALU) and logic gates configured to support operation of the processor 202. The network environment, such as, 100 may be accessed using the I/O interface 206 of the system 102. The I/O interface 206 may provide an interface for accessing various features and data stored in the system 102.

Pursuant to an example embodiment, the input module 202a is configured to receive input data. In an example, the input data may be received from, for example, the first computing device 106 and the second computing device 108. In certain other cases, the data of the first computing device 106 and the second computing device 108 may be stored in a database and retrieved therefrom. The input data may include a first set of simulated bioelectric signals 204a measured or collected by the first computing device 106 from an artificial brain or a head phantom, and patient bioelectric signals 204c measured or collected by the first computing device 106 from real patients or real human head. The input data may further include a second set of simulated bioelectric signals 204b measured or collected by the second computing device 108 from an artificial brain or a head phantom. For example, the bioelectric signals may be ultra-wideband time-domain measurements collected or measured by the first computing device 106 and the second computing device 108.

In an example, the first computing device 106 comprises a first antenna array to detect first scattering data. The first scattering data may correspond to the first set of simulated bioelectric signals 204a or the patient bioelectric signals 204c. Moreover, the second computing device 108 comprises a second antenna array to detect second scattering data that corresponds to the second set of simulated bioelectric signal 204b. For example, the first antenna array and the second antenna array are dual-comb microwave imaging sensors. For example, each of the first antenna array and the second antenna array consists of antennas, where each antenna serves as a sensor to receive signals.

In an example, the first antenna array and the second antenna array are implemented within a corresponding helmet. For example, the helmet may have an inner structure and an outer shell. The inner structure is a mechanical structure that may hold an antenna array, i.e., the first antenna array or the second antenna array. The antenna array may be positioned within the inner structure such that the antenna array may rest over the head of a patient or head phantom to measure bioelectric signals.

Continuing further, the measured bioelectric signals, i.e., the first set of simulated bioelectric signals 204a, the second set of simulated bioelectric signals 204b and the patient bioelectric signals 204c, may undergo several signal pre-processing steps to facilitate feature extraction. In this regard, the pre-processing module 202b is configured to pre-process the input data received from the first antenna array and the second antenna array. In an example, the pre-processing module 202b is configured to process time-domain input data using processing techniques to eliminate any delays resulting from variations in physical lengths of the radio-frequency cable connections.

Additionally, the pre-processing module 202b is configured to use reference signals to synchronize phase of each of the measured time-domain bioelectric signals.

Moreover, the measured time-domain bioelectric signals are scattered signals having scattering parameters. The measured time-domain bioelectric signals are signals that are scattered by different layers and composition (such as, white matter, gray matter, etc.) of the brain or the head phantom, as well as stroke conditions (such as tumor, hemorrhage, etc.). Further, the scattering parameters of the measured time-domain bioelectric signals describe properties of materials, i.e., human brain under test. For example, the scattering parameters may indicate how electromagnetic waves may propagate through the layers and composition of the brain.

In one example, the pre-processing module 202b may classify difference in scattering parameters during normal brain condition and stroke condition. In an example, such classification may be done based on ground-truth data or user feedback. In an example, the pre-processing module 202b may utilize Fourier transform to convert time domain bioelectric signals measured by a pair of antennas in the antenna array into frequency domain data. In an example, the pre-processing module 202b may denote a response between an antenna pair, i.e., a transmitting antenna j and a receiving antenna i, of an antenna array, for a fixed frequency wₖ, as sⱼᵢ (w). To ensure equalized power between the channels of the antenna pair, the response of each antenna pair of the antenna array at each frequency is normalized across one or more frequency dimensions. As the measured time-domain bioelectric signals exhibit a wide dynamic range caused due to the scattering parameters, the pre-processing module 202b is configured to normalize frequency-domain responses to a complex logarithm transformation. Subsequently, all values from a single measurement, such as the measurement taken by the first computing device 106 or the second computing device 108 at a particular time from a real, simulated, or artificial brain, are consolidated into a complex vector x∈C^{d}.

In an example, the elements of the data vector x are the elements of the set:

$$\{ log ( {s}_{ij} ( {w}_{k} ) / {c}_{ij} ) \vert k = 1 , … , {n}_{w} , 1 \leq i \leq j \leq n \} \qquad ( 1 )$$

where n is a number of frequencies chosen and

$${c}_{ij} = \sqrt{1 / n {\sum }_{k = 1}^{n} {\vert {s}_{ij} ( {w}_{k} ) \vert}^{2}} \qquad ( 2 )$$

In an example, the first antenna array or the second antenna array may include microwave-imaging based antennas on the inner side. For example, each antenna array may include 20 antennas, resulting in a total of 380 antenna pairs. As reciprocal antenna pairs have similar responses, a mean of each pair can be taken, resulting in 190 values at a given frequency. For example, a frequency in a range of 300 Mega Hertz (MHz) to 650 MHz may be used for measuring bioelectric signals, i.e., the first set of simulated bioelectric signals 204a, the second set of simulated bioelectric signals 204b and the patient bioelectric signals 204c. In an example, the first antenna array and the second antenna array may have a similar design, however, there may exist certain hardware differences due to circuit, error, components, etc. To this end, the measured bioelectric signals are pre-processed, for example, by eliminating delays from variations in physical lengths of the cable connections, using reference signals to synchronize phase of each of the measured time-domain bioelectric signals, and converting time-domain measurements into frequency-domain.

After pre-processing, the measured bioelectric signals are fed to the compensation module 202c. It may be noted, the present disclosure is based on a recognition that different hardware devices may cause significant data distribution shift among measurements obtained from these different devices. Due to the data shift, machine learning models, such as the classification neural network 104 as trained using data from one device may fail to generalize to data collected from another device. In other words, the classification neural network 104 trained on the first set of simulated bioelectric signals 204a and the patient bioelectric signals 204c measured by the first computing device 106 may fail to classify signals measured by the second computing device 108.

To address the aforementioned problem, the compensation module 202c is configured to generate a compensation factor 204d. In this regard, the compensation module 202c may generate the compensation factor 204d for the second computing device 108 based on a comparison between the first set of simulated bioelectric signals 204a and the second set of simulated bioelectric signals 204b. As the second computing device 108 is new, it may only be used on artificial or simulated brains (or artificial or simulated model of another anatomical part of a patient). Thereafter, a difference may be determined between the first set of simulated bioelectric signals 204a, i.e., signal data measured by the first computing device 106, and the second set of simulated bioelectric signals 204b, i.e., signal data measured by the second computing device 108.

According to an embodiment, the compensation module 202c is configured to generate the compensation factor based on a difference between the first set of simulated bioelectric signals 204a collected by the first computing device 106 and the second set of simulated bioelectric signals 204b collected by the second computing device 108. In an example, different types of signals may be passed through or generated within the artificial model of the brain. For example, these different types of signals may correspond to a particular part of brain, a particular nerve in the brain, a particular intensity of signal, etc. Furthermore, these different types of signals may be measured by both, the first computing device 106 and the second computing device 108. Further, the compensation module 202c may determine a difference between a first signal from the first set of simulated bioelectric signals 204a and a second signal from the second set of simulated bioelectric signals 204b. For example, the first signal and the second signal may relate to the same type. For example, based on the differences determined by the compensation module 202c, the compensation factor 204d may be determined. In an example, the compensation factor 204d may be generated based on an average of the differences.

Embodiments of the present disclosure are based on realizing that a difference, i.e., the compensation factor, between the first set of simulated signals 204a and the second set of simulated signals 204b is similar to or same as a difference between real patient bioelectric signals 204c and patient bioelectric signals that would be collected by the second computing device. To this end, based on the first set of simulated signals 204a and the patient bioelectric signals 204c measured by the first computing device 106 and the second set of simulated signals 204b measured by the second computing device 108, real patient bioelectric signals that would be collected by the second computing device may be inferred.

Thereafter, the processor 202, or the training module 202d is configured to generate compensated patient bioelectric signals based on the compensation factor 204d and the patient bioelectric signals 204c. In an example, the compensation factor 204d may indicate a degree of deviation or offset between signals measured by the first computing device 106 and signals measured by the second computing device 108. Based on the identified degree of deviation, the patient bioelectric signals 204c, i.e., real human data, collected by the first computing device 106 is compensated or updated. This compensated data closely matches reading that would be taken by the second computing device 108. Moreover, the first set of simulated bioelectric signals may also be compensated using the compensation factor 204d to make it suitable for generalizing or training the classification neural network 104 for the second computing device 108.

According to an embodiment, the alignment and compensation of patient bioelectric signals 204c aim to enhance the compatibility between the first computing device 106 and the second computing device 108. Initially, phases of the patient bioelectric signals 204c received from the first computing device 106 are aligned based on one signal from the patient bioelectric signals 204c thereby keeping phases of all of the patient bioelectric signals 204c same. Further, a signal from the first set of simulated signals 204a is aligned with a signal from the patient bioelectric signals 204c by keeping the phase same. Further, a signal from the second set of simulated signals 204b is aligned with a signal from the patient bioelectric signals 204c by keeping the phase same. For example, the signal from the first set of simulated signals 204a and the signal from the second set of simulated signals 204b may be aligned based on a same signal from the patient bioelectric signals 204c.

Thereafter, all the signals from the patient bioelectric signals 204c are compensated based on the compensation factor or the difference between the first set of simulated bioelectric signals 204a and the second set of simulated bioelectric signals. Further, the compensated patient bioelectric signals are inferred real head data for the second computing device 108. Continuing further, the training module 202d is configured to train the classification neural network 104. It may be noted, the classification neural network 104 is currently trained based on data collected by the first computing device 106 but it is not generalized for the second computing device 108. To this end, the training module 202d may feed the compensated patient bioelectric signals and the second set of simulated bioelectric signals 204b to the classification neural network 104 for training, re-training or finetuning. In this manner, the classification neural network 104 need not be trained from scratch, thereby reducing training cost and time.

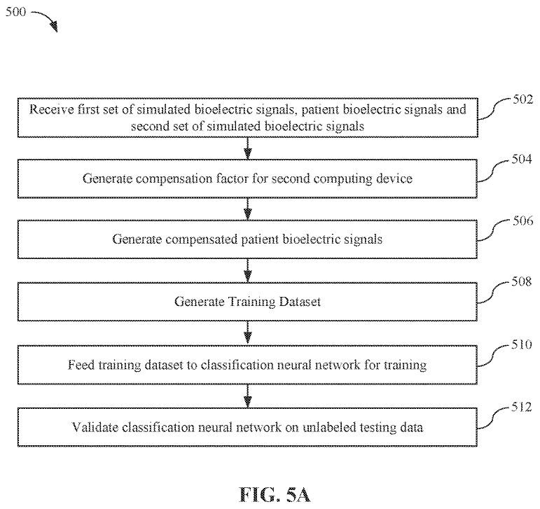

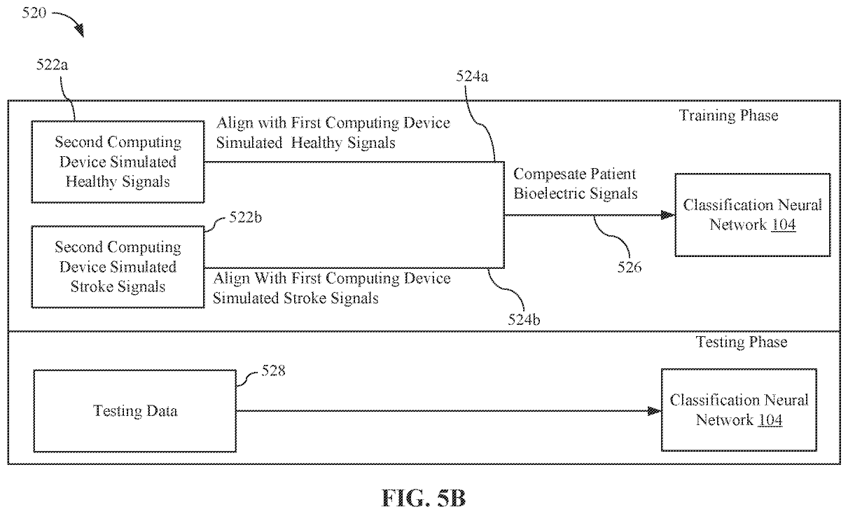

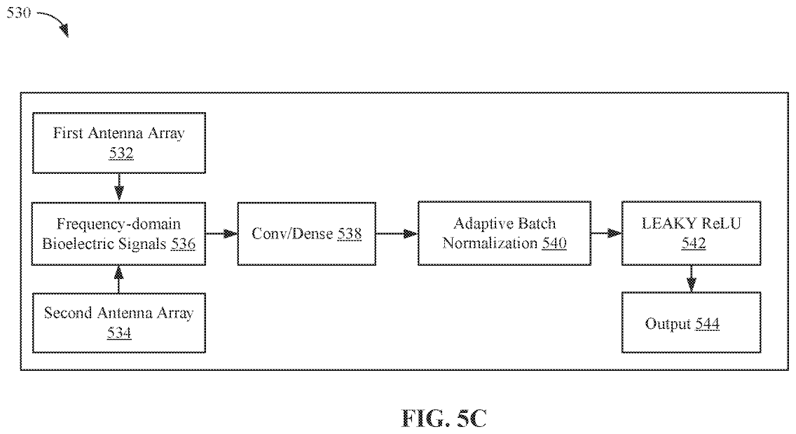

Moreover, as the classification neural network 104 is trained using both simulated data and real data that is compensated for the second computing device 108, the accuracy of the classification neural network 104 is improved significantly. After the training, the classification neural network 104 is deployed onto the second computing device 108 for collecting and classifying real human data or patient data. Details of training the classification neural network 104 are further described in conjunction with, for example, FIG. 5A, FIG. 5B, and FIG. 5C.

It may be noted that the present disclosure describes calculating compensation factor 204d for the second computing device 108 and further training the classification neural network 104 for deployment on the second computing device 108, however, this should not be construed as a limitation. Embodiments of the present disclosure may be utilized to generalize any neural network for any new device that does not have enough data by compensating data collected by an old device. For example, the classification neural network may also be generalized for a third computing device by compensating the data, i.e., the first set of simulated bioelectric signals 204a and the patient data bioelectric signals 204c, collected by the first computing device 106 and simulated signals collected by the third computing device. In certain cases, data collected by the second computing device 108 may also be compensated based on the simulated signals collected by the third computing device for generalizing the classification neural network 104 for the third computing device.

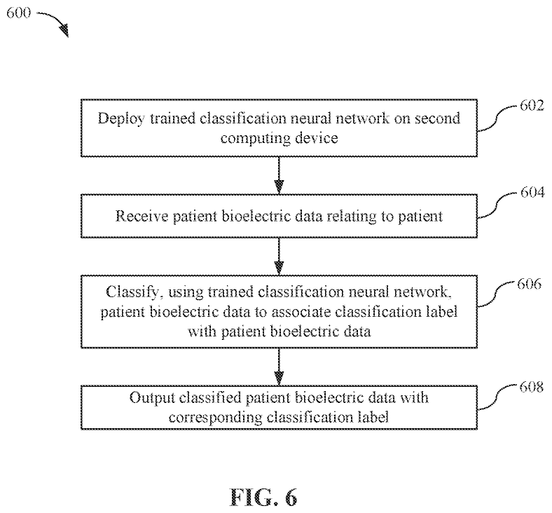

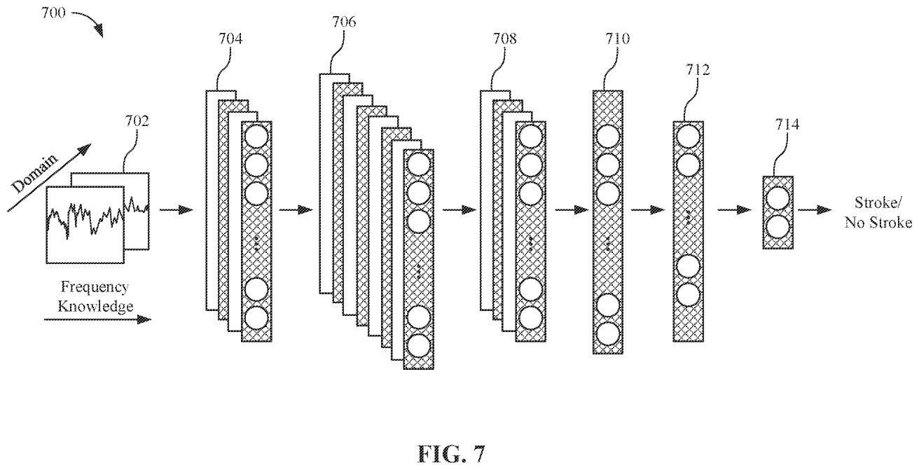

Details of the deployment of the trained classification neural network 104 on the second computing device 108 are described in conjunction with, for example, FIG. 6 and FIG. 7.

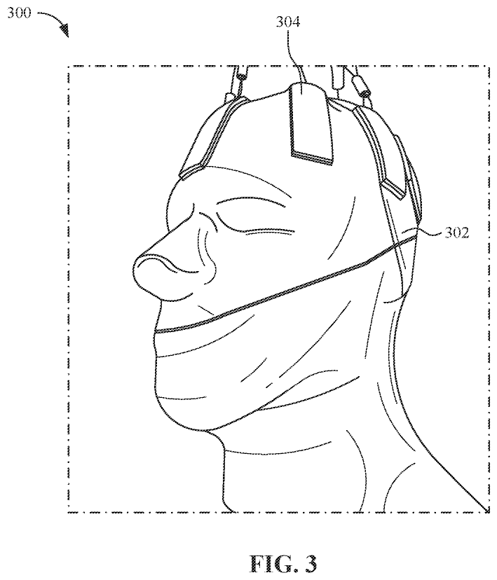

Referring to FIG. 3, there is shown a reference artificial brain model 300, in accordance with an example embodiment. Pursuant to the present example, the reference artificial brain model 300 is implemented as a physical head phantom 302. In an example, the head phantom 302 may be made from realistic tissue-mimicking materials.

In an example, the head phantom 302 acts as a reference and allows assessing source reconstruction procedures in electroencephalography and electrical stimulation profiles during transcranial electric stimulation. For example, the head phantom 302 can be used to simulate tomographic images of the head. Since the contribution of each tissue type to each voxel in the head phantom 302 is known, it can be used to test algorithms such as classification to identify parameters of brain waves based on each image voxel. Furthermore, since the same reference head phantom 302 may be used to collect the first set of simulated bioelectric signals 204a and the second set of simulated bioelectric signals 204b, this can be used to determine the compensation factor 204d accurately. In an example, the head phantom 302 is constructed or manufactured based on Ultrasound, MRI, X-Ray, CT scans of patients.

Further, an antenna array 304 may be positioned on top of the head phantom 302. The antenna array 304 may be the first antenna array of the first computing device 106 or the second antenna array of the second computing device 108. The antenna array 304 may collect data, i.e., simulated bioelectric signals from the head phantom 302. The first set of simulated bioelectric signals 204a may be measured by putting the first antenna array on the head phantom 302. Similarly, the second set of simulated bioelectric signals 204b may be measured by putting the second antenna array on the head phantom 302. For example, the antenna array 304 is used to measure electromagnetic signals or bioelectric signals emanating from or passing through the head phantom 302.

In an example, the head phantom 302 may be caused to mimic brain activities and brain waves of a healthy brain to collect healthy or normal condition measurements by the first antenna array and the second antenna array. Thereafter, a tube may be inserted into the head phantom 302 to simulate brain activities or brain waves of stroke for collecting stroke-related measurements. To this end, a first difference between measurements collected by the first antenna array and the second antenna array corresponding to healthy brain activity may be determined. Moreover, a second difference between measurements collected by the first antenna array and the second antenna array corresponding to stroke condition in the brain may be determined. Based on the determined differences, the compensation factor 204d is determined.

Although the present example describes the reference brain model as a physical head phantom, it should not be construed as a limitation. In other examples, the reference brain model may be implemented as a computer simulation.

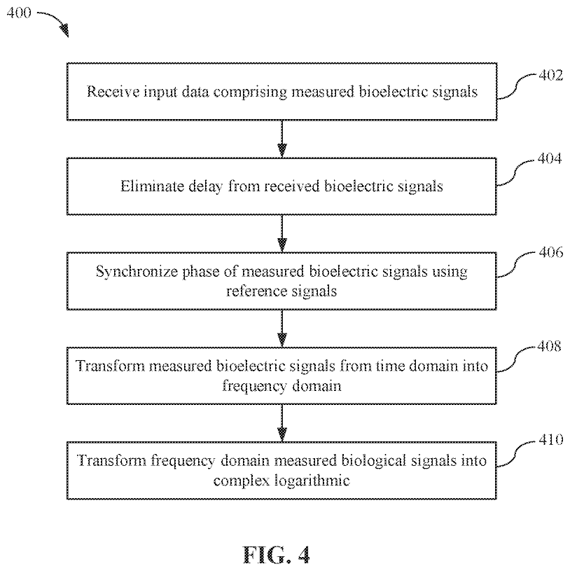

FIG. 4 illustrates a flow chart 400 of a method for pre-processing measured bioelectric signals, in accordance with an embodiment. The pre-processing module 202b is configured to pre-process the measured bioelectric signals, such as the first set of simulated bioelectric signals 204a, the second set of simulated bioelectric signals 204b, and the patient bioelectric signals 204c. The flowchart 400, as depicted, outlines a structured sequence of operation carried out by the pre-processing module 202b.

At 402, input data is received. The input data includes measured bioelectric signals, i.e., the first set of simulated bioelectric signals 204a and the patient bioelectric signals 204c measured by the first computing device 106 and the second set of simulated bioelectric signals 204b measured by the second computing device 108. For example, the first computing device 106 and the second computing device 108 may include antenna array 304 comprising antennas to emit microwave signals that may be bombarded onto the head phantom 302 or heal human head and receive reflected signals. The reflected signals are measured as the first set of simulated bioelectric signals 204a, and the patient bioelectric signals 204c or the second set of simulated bioelectric signals 204b.

At 404, delay is eliminated from the received measured bioelectric signals. In order to ensure accuracy and reliability of the measured bioelectric signals, presence of delays introduced due to variations in physical lengths of the radio-frequency cable connections in the antenna array 304 is eliminated. These delays may distort a temporal alignment of measured signals from different antennas and/or antenna pairs. In this regard, for example, the measured input data is analyzed to make precise adjustments to compensate for the variations in cable lengths. This results in synchronized time-domain measured signals across all antennas and/or antenna pairs.

At 406, phase of measured bioelectric signals of the input data are synchronized. In this regard, one or more reference signals may be used to synchronize phase of each of the measured bioelectric signals from the first set of simulated bioelectric signals 204a, the patient bioelectric signals 204c and the second set of simulated bioelectric signals 204b. For example, by comparing phase and timing of the measured bioelectric signals with the reference signals, any deviations or discrepancies in phase are identified and rectified.

At 408, the measured bioelectric signals are transformed from time-domain to frequency-domain. In this regard, Fourier transform may be performed on the time-domain measured bioelectric signals to convert the measured bioelectric signals into frequency-domain.

At 410, the frequency-domain measured bioelectric signals are transformed to complex logarithmic. In an example, the complex logarithm transformation is applied to normalize the frequency-domain measured bioelectric signals. In this manner, all values from a single measurement of the first set of simulated bioelectric signals 204a, the patient bioelectric signals 204c and the second set of simulated bioelectric signals 204b are consolidated into a complex vector x∈C^{d}. The elements of the data vector x are the elements of the set defined by Equation (1). After pre-processing the frequency-domain measured bioelectric signals, i.e., the first set of simulated bioelectric signals 204a and the second set of simulated bioelectric signals 204b, are compared to determine the compensation factor 204d.

FIG. 5A illustrates a flowchart 500 of a training process of the classification neural network 104, in accordance with one or more example embodiments. FIG. 5B illustrates an exemplary block diagram 520 of a training process of the classification neural network 104. For sake of brevity, the elements of the FIG. 5A and FIG. 5B are described in conjunction.

At 502, the first set of simulated bioelectric signals 204a and patient bioelectric signals 204c are received from the first computing device 106. Moreover, the second set of simulated bioelectric signals 204b are received from the second computing device 108. In an example, the first set of simulated bioelectric signals 204a comprises a set of signals corresponding to simulated healthy brain activity (referred to as, first computing device healthy signals) and a set of signals corresponding to simulated stroke brain activity (referred to as, first computing device stroke signals). Similarly, the second set of simulated bioelectric signals 204b comprises a set of signals corresponding to simulated healthy brain activity (referred to as, second computing device healthy signals 522a) and a set of signals corresponding to simulated stroke brain activity (referred to as, second computing device stroke signals 522b).

In an example, both the first computing device 106 and the second computing device 108 are utilized to measure the bioelectric signals on a common reference or artificial brain model, such as the head phantom 302. The head phantom 302 represents a standardized reference, enabling to quantify and compensate for measurement differences between the first computing device 106 and the second computing device 108.

In accordance with an embodiment, the first set of simulated signals 204a may include first computing device simulated healthy signals and first computing device simulated stroke signals measured by the first computing device 106 using the head phantom 302. Similarly, the patient bioelectric signals 204c may include first computing device patient healthy signals and first computing device patient stroke signals measured by the first computing device 106 using a real human head.

At first, the first computing device simulated healthy signals and the first computing device simulated stroke signals are aligned with first computing device patient healthy signals and first computing device patient stroke signals, respectively. For example, the alignment is performed based on a correlation between two data samples, such as the first computing device simulated healthy signals (A) and the first computing device patient healthy signals (B). For example, based on this alignment, a shift in, say, the first computing device simulated healthy signals (A) is determined so that it aligns or matches best with phase of the first computing device simulated healthy signals (B). For example, the alignment may be performed using a sliding window step-by-step. At each step, a level of similarity in phase between (A) and (B) may be determined. The level of similarity may be calculated using an inner product (or dot product) of the two signals (A) and (B). When the level of similarity is at its highest, it is understood that (A) and (B) align well. To this end, for every possible shift k of (A), the inner product of (A) and (B) is calculated based on:

$$C ( k ) = {\sum }_{n}^{A} ( n + k ) \cdot B ( n ) \qquad ( 3 )$$

The goal is to find the shift k where C (k) is maximum. In an example, C (k) may be calculated for every possible shift k of (A). Further, the k value where C (k) is at its maximum is identified. This k value would correspond to the best alignment between (A) and (B). In this manner, (A) and (B) are aligned. To this end, the first computing device patient healthy signals and the first computing device patient stroke signals, are aligned with the first computing device simulated healthy signals and the first computing device simulated stroke signals, respectively.

Further, the second set of simulated bioelectric signals 204b are aligned with the first set of simulated bioelectric signals 204a. In one example, the second set of simulated bioelectric signals 204b includes second computing device simulated healthy signals 522a and second computing device simulated stroke signals 522b. Further, the second computing device simulated healthy signals 522a are aligned, at 524a, based on the first computing device simulated healthy signals. Similarly, the second computing device simulated stroke signals 522b are aligned, at 524b, based on the first computing device simulated stroke signals.

For example, a difference between the aligned first computing device simulated healthy signals and the second computing device simulated healthy signals 522a may be determined. Similarly, a difference between the aligned first computing device simulated stroke signals and the second computing device simulated stroke signals 522b may be determined. In this manner, For example, the differences are used to compensate for the variations in the patient bioelectric signals 204c, i.e., the first computing device patient healthy signals and the first computing device patient stroke signals.

At 504, the compensation factor 204d is generated for the second computing device 108. In an example, by comparing the measurements obtained from the second computing device 108 and the first computing device 106 on the reference head phantom 302, differences between collected signals by the two devices are identified.

At 506, compensated patient bioelectric signals are generated based on the compensation factor 204d and the patient bioelectric signals 204c. For example, based on the differences between the second computing device simulated healthy signals 522a and the first computing device healthy signals, as well as the second computing device simulated stroke signals 522b and the first computing device stroke signals, the compensation factor 204d is determined to compensate the patient bioelectric signals 204c, as shown in 526.

To this end, by employing compensation techniques using the reference head phantom 302, device variations between the second computing device 108 and the first computing device 106 are determined. The compensation of the patient bioelectric signals 204c based on the measurement differences, i.e., the compensation factor 204d, ensures that actual measurements collected from different devices are adjusted to reduce the impact of variations during training. Subsequently, the compensated patient bioelectric signals 204c are passed through a feature construction module for further processing and analysis.

For example, a compensation between the aligned the first set of simulated bioelectric signals 204a and the second set of simulated bioelectric signals 204b may be denoted as:

$${X}_{{l}_{1}}^{′} = {X}_{{l}_{1}} + ( {X}_{{j}_{2}} - {X}_{{j}_{1}} ) \qquad ( 4 )$$

where X′ₗ_{₁}, is a compensated patient bioelectric signal from the patient bioelectric signals 204c from the first computing device 106, Xₗ_{₁}, is patient bioelectric signal from the patient bioelectric signals 204c before compensation, Xⱼ_{₁}, is the aligned simulated bioelectric signals from the first set of simulated bioelectric signals 204a, and Xⱼ_{₂}, is simulated bioelectric signals from the second set of simulated bioelectric signals 204b from the second computing device 108. After compensation, the X′ₗ_{₁}, will closely match to Xₗ_{₂}, for the second computing device 108. For example, the X′ₗ_{₁}, is used to train the classification neural network 104 for the second computing device 108.

Thereafter, at 508, a training dataset is generated. In an example, the training dataset may include compensated patient bioelectric signals 204c, and second set of simulated bioelectric signals 204b. For example, the training dataset may be divided into two parts, one, for training of the classification neural network 104, and second for testing the classification neural network 104.

At 510, the training data or a portion of the training data is fed to the classification neural network 104 for training. For example, during the training, the classification neural network 104 may extract features of the received training data and learn patterns of healthy and stroke conditions in the bioelectric signals in the training data.

In an example, scattering of microwaves caused by wide-band antenna arrays, such as the antenna array 304 can be indicative of a presence of an anomaly or a disease. Further, the anomaly or the disease can be used to obtain dielectric signature of an affected area in the brain. With the aid of the classification neural network 104, anomaly, such as strokes can be detected and various details related to the stroke, such as its type (hemorrhagic or ischemic) and the affected area of the brain (left or right), etc., can be predicted. In an example, Convolutional Neural Network (CNN) is used to design the classification neural network 104 to analyze the information of the bioelectric signals across the different devices through alignment and compensation. This may provide a K-fold average accuracy of 91.5% for classification of bioelectric signals.

Further, at 512, the classification neural network 104 is validated. In an example, a predicted output of classification neural network 104 for testing data is validated to validate operation of the classification neural network 104. For example, the testing data may be a part of the training dataset. The testing data may be unlabeled and may be used to cause the classification neural network 104 to predict outcomes. For example, the outcome may be to associate bioelectric signals form the testing data with a classification label. For example, the classification label may correspond to (i) presence of stroke, (ii) absence of stroke.

Referring to FIG. 5B, the classification neural network 104 may further be tested after the training. In this regard, the testing data 528 comprising the compensated patient bioelectric signals, the second set of simulated bioelectric signals 204b, a combination thereof, or a portion thereof may be fed to the classification neural network 104. The testing data may not include labels corresponding to bioelectric signals. Further, during the testing process, the testing data is used to evaluate the proficiency of the classification neural network 104 in analyzing, predicting, and classifying classification labels.

Further, the trained and validated classification neural network 104 is deployed on the second computing device 104. The process allows the second computing device 108 to utilize the classification neural network 104 for the specific task of classifying patient bioelectric data measured or collected by the second computing device 108.

Referring to FIG. 5C, a block diagram 530 for training the classification neural network 104 is illustrated, in accordance with an example embodiment. In an example, an architecture of the classification neural network 104 may include a plurality of one-dimensional (1D) convolution neural networks (CNNs). To this end, pre-processed frequency-domain bioelectric signals 536 are obtained from a first antenna array 532 of the first computing device 106 and a second antenna array 534 of the second computing device 108. It may be noted, the bioelectric signals from the first antenna array 532 of the first computing device 106 are pre-processed and compensated, i.e., compensated patient bioelectric signals 204c, whereas the bioelectric signals, i.e., second set of simulated bioelectric signals 204b, from the second antenna array 534 of the second computing device 108 are pre-processed. The architecture of the classification neural network 104 is further described in detail in conjunction with FIG. 7.

According to the present embodiment, the antenna arrays, i.e., the first antenna array 532 and the second antenna array 534, are configured to sense microwave signals. In an example, a plurality of antenna sensors, say 20 to 30 antenna sensors, may be positioned within the antenna arrays 532 and 534. The plurality of antenna sensors may be dual-comb microwave imaging-based sensors. Further, the computing devices, such as the first computing device 106 and the second computing device 108 housing the first antenna array 532 and the second antenna array 534, respectively, may perform processing of radio-frequency signals based on dual-comb microscopy principles. For example, after microwave signals are sensed by the antenna arrays 532 and 534, the computing devices 106 and 108 may perform dual-comb microwave signal processing techniques on the measured signals.

In an example, the antenna arrays 532 and 534 or the antenna sensors may sense or measure scattering parameters of the microwave signals reflected from the artificial reference brain models or patient brains to collect bioelectric signals. The scattering parameters (or S-parameters) may indicate electrical behavior of linear electrical networks within brain when undergoing various steady state stimuli by electrical signals transmitted to the real or artificial brain. In an example, the scattering parameters may be measured with a vector network analyzer. In another example, the reflected microwave signals or the bioelectric signals are measured or sensed as wide-band time domain bioelectric signals. Further, frequency components of wide-band time domain bioelectric signals can be used to approximate the scattering parameters for the bioelectric signals.

Based on the collected bioelectric signals, complex vectors consisting of complex values may be generated. For example, as the antenna arrays 532 and 534 include a plurality of antenna sensors, each pair of antenna sensors may provide one measurement or a complex value at a corresponding frequency point. To this end, a complex vector for a particular antenna array, say, the first antenna array 532, would include complex measurements or values collected or measured by the first antenna array 532 over all antenna pairs and all investigated frequencies. For example, every measurement (from an antenna pair) of the first antenna array 532 is stored as a complex value with real and imaginary parts in a complex vector corresponding to the first computing device 106 or the first antenna array 532. In an example, the complex vector is used as a feature vector for the input of the classification neural network 104.

In an example, the measured or received bioelectric signals are fed to convolution dense (Conv/Dense) layers 538, i.e., convolution fully connected feedforward CNN layers, of the classification neural network 104. In an example, the Conv/Dense layers 538 are configured to perform feature extraction by convolution kernels. For example, a data sample or an input bioelectric signal, i, in the format of 1-D array is processed by convolutional kernels in each of the Conv/Dense layers 538 as follows:

$$c [ i ] [ {k}_{out} ] = b [ {k}_{out} ] + {\sum }_{{k}_{i n} = 1}^{K} w [ {k}_{out} ] [ {k}_{i n} ] \cdot I [ i ] [ {k}_{i n} ] \qquad ( 5 )$$

where ‘⋅’ is the convolution operator, kᵢₙ and kₒᵤₜ is the index of input and output channels, I is the input signal value, K is the total number of convolution kernels for all input channels, and w and b are weight and bias in the corresponding channel.

The Conv/Dense layers 538 or fully connected layers connect all neurons from one or more previous layers to all neurons in a current layer. For each neuron in the current layer, it calculates a weighted sum of inputs from the one or more previous layers, adds a bias term, and applies an activation function as follows:

$$c [ k ] = {\sum }_{n}^{N} w [ k ] [ n ] \cdot I [ k ] [ n ] + b [ k ] \qquad ( 6 )$$

where I is the input, w and b are the weights and bias in the corresponding layer, and N is a number of input size.

Further, adaptive batch normalization layers 540 and non-linear activation functions (such as Leaky ReLU) layers 542 are applied within the multiple layers of the CNNs to improve the convergence and generalization of the classification neural network 104. For example, the adaptive batch normalization layers 540 and the Leaky ReLU activation function layers 542 are applied after each fully connected layer of the CNN. Moreover, dropout layers with a predefined dropout rate (for example, 15% dropout rate, 20% dropout rate, 30% dropout rate, etc.) are applied after each convolutional and fully connected CNN layer of the classification neural network 104 to prevent overfitting. In addition, an output layer 544 is a SoftMax and classification layer, which predicts a probability distribution and a probability score of each bioelectric input signal belonging to one of the classes of classification labels corresponding to healthy brain activity and unhealthy, stroke or anomalous brain activity.

The adaptive batch normalization layers 540 are used in the classification neural network 104 to address the domain shift between signals collected by different devices. In particular the adaptive batch normalization (AdaBN) layers 540 allows the classification neural network 104 to adapt to new distributions of input data during inference.

During the training phase, the compensated patient bioelectric signals 204c and the second set of simulated bioelectric signals 204b are fed to one or more adaptive batch normalization layers 540 of the classification neural network 104. The adaptive batch normalization layers 540 are trained to learn mean and variance corresponding to the compensated patient bioelectric signals 204c.

As may be noted, the first antenna array 532 of the first computing device 106 and the second antenna array 534 of the second computing device 108 may measure bioelectric signals independently. To this end, to ensure comparability and facilitate analysis, Z-transform may be applied to both sets of measured data to standardize them. For example, the Z-transform may be applied to the second set of simulated bioelectric signals 204b and the compensated patient bioelectric signals 204c for standardization. This Z-transformation involves adjusting each data from the second set of simulated bioelectric signals 204b and the compensated first set of simulated bioelectric signals 204a using the mean and the variance to normalize the training dataset. The adjustments for the mean and the variance may be identified using the following formula:

$$Z = ( X - μ ) / σ \qquad ( 7 )$$

where Z represents a transformed data point, X represents an original data point collected from the respective computing device, μ is a mean value of the data collected from that computing device, and σ is the standard deviation of the data collected from that computing device. By performing the Z-transform separately for each of the computing devices, the first set of simulated bioelectric signals 204a and the second set of simulated bioelectric signals 204b are flattened and standardized, making it easier to compare and analyze data from both the computing devices 106 and 108. The standardized data, i.e., the standardized first set of simulated bioelectric signals 204a and the standardized second set of simulated bioelectric signals 204b allows for more effective and reliable comparisons for generating the compensation factor 204d.

Once the mean and the variance for normalization are learnt, then, during an inference phase, the adaptive batch normalization layers 540 dynamically adjusts mean and variance of the current batch of input signal collected by, for example, the second antenna array 534 of the second computing device 108. As a result, performance and robustness of the classification neural network 104 in handling variations in input data is improved. The adaptive batch normalization layers 540 may enable the classification neural network 104 to effectively generalize to the bioelectric signals collected from the second computing device 108, i.e., the new device, even when there are significant differences between the distribution of bioelectric signals from the first computing device 106 and the second computing device 108.

In an embodiment, during the training phase, the classification neural network 104 is trained using a plurality of simulated healthy brains and a plurality of simulated strokes collected by the first computing device 106. Further, during the testing phase, the classification neural network 104 is tested on a plurality of simulated healthy brains and a plurality of simulated strokes measured by the second computing device 108.

In an example, an Adaptive Moment Estimation optimizer (Adam optimizer) model may be used for the optimizing the classification neural network 104 during its training, testing and/or re-training. For example, the Adam optimizer is configured to update weights of the classification neural network 104. The Adam optimizer may be implemented with a batch size of 100 and 30 epochs of training. In an example, an initial learning rate for operation of the Adam optimizer may be set to 0.01 and a drop rate is set to 1% for every 5 epochs. Moreover, binary cross-entropy loss is used to calculate a difference between the predicted classification labels and ground truth classification labels. During testing, to evaluate the performance of classification neural network 104, K-fold validation technique with 5-10 splits may be used.

During the testing phase, a classification loss and performance of the 1-D CNN-based classification neural network 104 are determined. In an example, a true positive rate (TPR), a false alarm rate (FAR), a classification accuracy (ACC), and a receiver-operating characteristic (ROC) are calculated to show the classification performance of the classification neural network 104. The classification loss and the classification performance are used to ensure high accuracy and sensitivity in stroke detection.

The TPR indicates a performance metric used to evaluate the effectiveness of the classification neural network 104. The FAR is calculated based on a number of false positives (FP) and a number of true negatives (TN), such that (FP+TN) a total number of true negatives. FAR is calculated as FP/FP+TN to indicate a probability that a false alarm will be raised, i.e., a positive result will be given when a true value is negative. The ACC indicates a metric for evaluating performance of the classification neural network 104. The ACC may be calculated based on a number of correct predictions and a total number of predictions. Moreover, the ROC may be a graph or a curve that shows the performance of the classification neural network 104 at all classification thresholds. The ROC may be plotted based on a true positive rate and a false positive rate of the classification neural network 104.

To this end, the aforementioned performance metric may be calculated for the training phase and the testing phase to evaluate performance of the classification neural network 104. Based on the evaluation, the weights of the classification neural network 104 may be adjusted and/or the classification neural network 104 may be re-trained. In certain cases, for re-training, an updated labeled set of bioelectric signals is generated that may include one or more bioelectric signals.

It an example, a classification loss after a predefined number of iterations, say 20, during the training phase is compared with a classification loss after the same predefined number of iterations during the testing phase. Pursuant to present disclosure, a high average TPR, for example, TPR of 99%, is achieved both during the training phase and the testing phase. Moreover, a low FAR, for example, FAR in a range of 2% to 5%, is achieved. Furthermore, high ACC, for example, ACC in a range of 95% to 99%, is also achieved using the classification neural network 104 trained based on embodiment described in the present disclosure.

Referring to FIG. 6, a flowchart 600 of a method for implementing the classification neural network 104 is provided, in accordance with an example embodiment. The trained classification neural network 104 is deployed on the second computing device 108. Further, the trained classification neural network 104 is configured to classify signal waves of the patient in one of the two classification labels, i.e., healthy or stroke.

In this regard, at 602, patient bioelectric data is received. The patient bioelectric data is collected using the second antenna array 534 of the second computing device 108. The patient bioelectric data may relate to an anatomical part, such as the brain of a patient. The patient bioelectric data includes one or more signal waves. The one or more signal waves may correspond to different frequencies and/or different parts of the anatomical part of the patient.

Further, at 604, the trained classification neural network 104 is deployed on the second computing device 108. The second computing device 108 includes the second antenna array 534 for collecting the patient bioelectric data.

For example, the classification neural network 104 trained on the second set of simulated bioelectric signals 204b and the patient bioelectric signals 204c that is compensated based on the compensation factor 204d.

At 606, the patient bioelectric data is classified using the classification neural network 104. In this regard, a classification label or at least one classification label is assigned to each of the one or more signal waves using the trained classification neural network 104. In particular, the trained classification neural network 104 is configured to predict a probability score across each of different classification labels for classifying a signal wave from the patient bioelectric data. Subsequently, the trained classification neural network 104 may predict a classification label corresponding to the signal wave based on the probability scores across the different classification labels. To this end, each of the different signal waves of the patient bioelectric data may be classified, i.e., assigned a classification label. In certain cases, the patient bioelectric data may include a single signal wave. In such a case a single classification label may be assigned to the signal wave, or different segments of the signal wave may be classified to assign a classification label. For example, with regard to the patient bioelectric data relating to brain signals, the classification label indicates a presence, or an absence of a health condition, such as stroke, associated with the brain of the patient.

At 608, classified patient bioelectric data along with corresponding one or more classification labels is output. For example, the trained classification label may predict classification labels for each of the signal waves or different segments of a single signal wave, or a single classification label for the patient bioelectric data. Based on the prediction, the second computing device 108 may cause to display the classified patient bioelectric data within a user interface. In this regard, a display of the second computing device 108 or other display accessible to the second computing device 108 may be used to display the patient bioelectric data or the signal waves along with corresponding classification label(s). In other cases, the classification label(s) corresponding to the patient bioelectric data is fed to another downstream system for further processing of the patent bioelectric data.

FIG. 7 illustrates a schematic diagram 700 of an architecture of the classification neural network 104, in accordance with an example embodiment of the present disclosure. The embodiments of the present example are explained with regard to implementation or inference phase of the trained classification neural network 104.

It may be noted, the CNN-based classification neural network 104 includes an input layer 702, one or more adaptive batch normalization layers, one or more feature extraction layers (depicted as layers, 704, 706, 708, 710 and 712), and an output layer 714.

The input layer 702 may receive input data, such as patient bioelectric data measured by the second antenna array 534 of the second computing device 108. The patient bioelectric data may include one or more signal waves measured from different parts of an anatomical part, such as the brain of a patient. For example, the measured patient bioelectric data may be pre-processed. In an example, the input data may be a frequency signal with a dimension of, for example, 2×3185 frequency points. Each of the two channels of a frequency signal may represent real and imaginary parts of a signal wave of the measured patient bioelectric data. Further, both channels would have 3185 frequency points.

The input frequency signal is then processed through three 1-D convolutional layers 704, 706, and 708, each with 48, 96, and 48 kernels (1×7), respectively. Moreover, batch normalization using the one or more batch normalization layers and non-linear activation functions (such as, Leaky ReLU) are applied after each of the 1-D convolutional layers 704, 706, and 708 to improve the convergence and generalization of the classification neural network 104.

It may be noted, the one or more adaptive batch normalization layers may have learnt mean and variance during training phase. To this end, during the implementation, the one or more batch normalization layers may perform adaptive batch normalization on the patient bioelectric data based on the learnt mean and variance It may be noted, the mean and the variance are learnt, at first, based on the compensated patient bioelectric signals 204c collected by the first computing device 106 and the second set of simulated signals 204b used during the training phase. Further, based on the testing data 528, a domain shift may occur causing to change the mean and the variance.

Some embodiments of the present disclosure are based on a realization that by training the classification neural network 104 for deployment on the second computing device 108 using the compensated patient bioelectric signals 204c improves the accuracy of the classification neural network 104 significantly. However, an objective of the present disclosure is to further improve the accuracy of the classification neural network 104.

Some embodiments of the present disclosure are based on a realization that one of the reasons for the low readiness rate of the classification neural network 104 may come from the normalization layer(s).

Some embodiments are based on a realization that the batch normalization layer(s) may use the mean and the variance of the training dataset learnt based on the compensated patient bioelectric signals 204c and the second set of simulated bioelectric signals 204b to normalize the input data, i.e., the measured patient bioelectric data. However, the mean and the variance of the testing data 528 may be different from the mean and the variance of the training data, resulting in domain shift of the batch normalization layer(s) of the classification neural network 104. As a result, the trained and validated classification neural network 104 may fail to operate accurately on the input data for normalization.

An objective of the present disclosure is to use adaptive batch normalization technique in the batch normalization layer(s) to account for the domain shift caused due to the training data and the testing data 528. In this regard, after the trained classification neural network 104 is deployed on the second computing device 108, a mean and a variance of the data, i.e., the patient bioelectric data, collected by the second computing device 108 is used to normalize subsequent data that would be collected by the second computing device 108. As time goes by, and the amount of data collected and processed by the second computing device 108 increases, the mean and the variance of the batch normalization layer(s) will gradually stabilize. Subsequently, the classification neural network 104 or the batch normalization layer(s) will also stabilize.

After the processing of the frequency-domain input patient bioelectric data by the three 1D convolution layers 704, 706 and 708, generated resulting feature maps are flattened and passed through two fully connected layers, depicted as one or more feature extraction layers 710 and 712. The one or more feature extraction layers 710 and 712 are downsized to 1024 and 512 kernels, respectively, to extract high-level features from the input patient bioelectric data. These one or more feature extraction layers 710 and 712 are configured to, for example, represent the extracted features in a feature vector. The one or more feature extraction layers 710 and 712 may be implemented using several convolution layers followed by max-pooling and an activation functions.

In addition, the classification neural network 104 includes dropout layers with a predefined dropout rate, say 30% dropout rate, applied after each convolutional layer 704, 706 and 708 and fully connected layer 710 and 712 to prevent overfitting.

After the features of the input patient bioelectric data are extracted, the extracted features are used by the output layer 714 to predict a probability distribution of the input patient bioelectric data belonging to one of a number of different classification label classes. For example, the classification label classes learnt during the training may correspond to “healthy brain activity” and “stroke or unhealthy or anomalous brain activity”. In an example, the output layer 714 is configured to generate a probability score for each of the different classification labels such that a sum of the probability scores for each of the different classification labels is 1. Based on the probability scores, the output layer 714 outputs a predicted classification label for the input patient bioelectric data. Particularly, the classification label having higher probability score is selected as the predicted classification label for the input patient bioelectric data. For example, the output layer 714 is implemented using a SoftMax layer. Moreover, the output layer 714 may include two layers corresponding to two classification labels, namely, healthy and stroke.

The output layer 714 further outputs classified patient signal data based on the probability scores. The classified patient signal data comprises at least one classification label assigned to the patient bioelectric data. In an example, a classification label may be assigned to each of the one or more signal waves of the patient bioelectric data. The classification label assigned to the patient bioelectric data may indicate if any part of the brain of the patient has stroke conditions or not.

In this manner, different signals collected from the same or different patients may be classified. During a scanning procedure of a patient, the multiple signals may be collected from different parts of the brain of the patient. These different signals may be classified to analyze which part of the brain indicates stroke and which is healthy. Subsequently, identification of stroke condition and localization of the stroke condition in the brain may be done using the classification label(s) assigned to the patient bioelectric data.

It may be noted that a number of layers, a number of kernels, etc. as described in the present example is only exemplary. A person with ordinary skill in the art would be able to recognize different architectures of classification neural networks.

In addition, the input patient bioelectric data or the one or more signal waves collected by the second computing device 108 corresponds to brain waves of a patient. To this end, the classification label classifying the input signal corresponds to a presence of stroke condition associated with the brain of the patient, i.e., unhealthy, stroke or anomalous brain activity; or an absence of stroke condition associated with the brain of the patient, i.e., healthy brain activity. The use of the classification neural network 104 in devices or systems for stroke detection is only exemplary and should not be considered as limiting in any way. The classification neural network 104 may be trained on other physiological data for generalizing new devices for monitoring or classifying other types of physiological data.

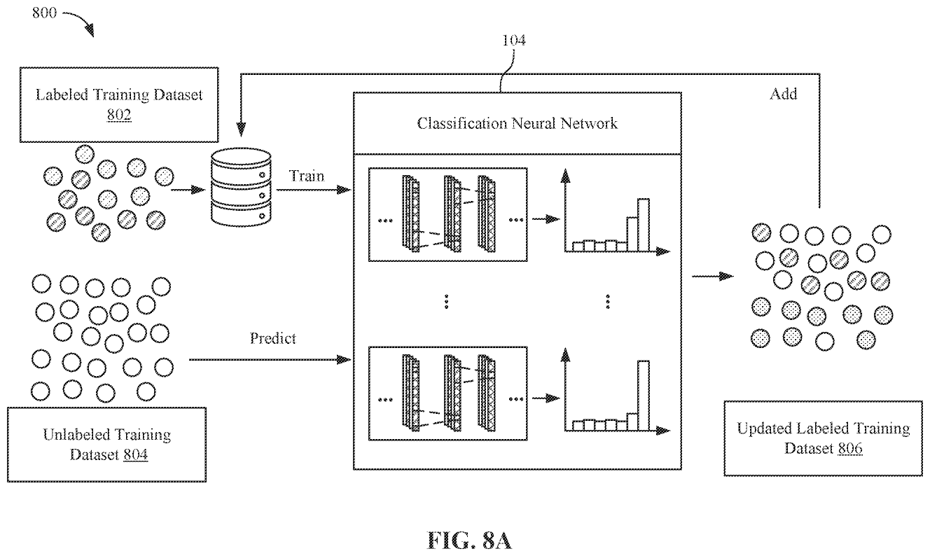

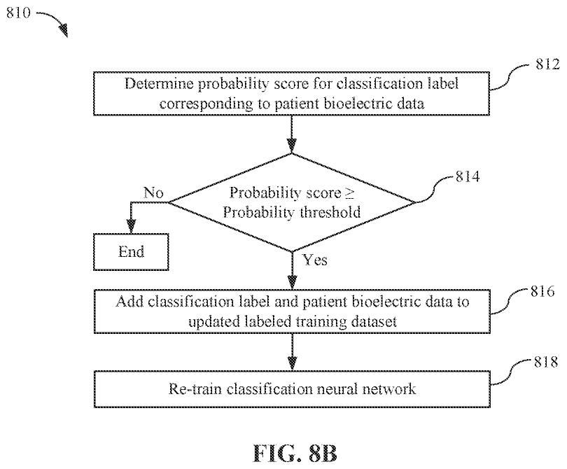

FIG. 8A illustrates an example schematic diagram 800 of a re-training process of the classification neural network 104, according to an example. The re-training is performed to further improve accuracy of the classification neural network 104 after its deployment on the second computing device 108.

For example, an accuracy of the classification neural network 104 after the training of the classification neural network 104 on the compensated patient bioelectric signals 204c and using adaptive batch normalization techniques in the batch normalization layers after the deployment on the second computing device 108 is increased substantially. For example, the accuracy after the training and the adaptive batch normalization techniques may be in a range of 85-90%.

In this regard, the classification neural network 104 is trained on a labeled training dataset 802 comprising the compensated patient bioelectric signals 204c and the second set of simulated signals 204b. Furthermore, the classification neural network 104 is validated or tested on unlabeled training dataset 804 comprising the testing data 528. Thereafter, the classification neural network 104 is deployed on the second computing device 108 to classify the patient bioelectric data collected from a patient.

In this regard, the trained classification neural network 104 may classify the patient bioelectric data based on probability scores for the patient bioelectric data corresponding to each of the different classification labels. For example, if the classification label “presence of stroke” has a higher probability score, say 0.8, than the classification label “absence of stroke”, say 0.2, for the patient bioelectric data, then the classification label “presence of stroke” is assigned to the patient bioelectric data and the classified patient bioelectric data is provided as output.

To this end, the objective of the present disclosure is to further improve the accuracy of the classification neural network 104. In this regard, after the classification neural network 104 is deployed on the second computing device 108 and the second computing device 108 is put to use on real patients, the accuracy is improved. For example, the accuracy of the deployed classification neural network 104 is improved based on patient bioelectric data relating to different patients collected by the second antenna array 534 of the second computing device 108.

Pursuant to the present example, during the inference phase, the classification neural network 104 is configured to assign an outcome, i.e., a predicted classification label, that it considers to be the most appropriate to each patient bioelectric data collected from different patients.

Some embodiments of the present disclosure are based on a realization that after the deployment, data measured by the second computing device 108 may be used to improve the accuracy. However, during the use of the second computing device 108, labeling of the measured data by experts, such as doctors, nurses, clinicians, or any medical practitioner is not feasible.

Subsequently, after the second computing device 108 is put to use, a large amount of data, i.e., patient bioelectric data form multiple patients, is received but there will be no label associated with the measured data. To further train the classification neural network 104 on the measured patient bioelectric data, a pseudo-labeling technique is utilized. In this regard, using a result of the classification neural network 104 for the currently measured patient bioelectric data, an updated labeled training dataset 806 is generated. For example, based on probability scores for classification labels assigned to a pool of patient bioelectric data of multiple patients, certain patient bioelectric data from the pool having high probability scores are added to the updated labeled training dataset 806. For example, if a probability score for a classification label for patient bioelectric data of a patient is greater than a probability threshold, then the patient bioelectric data along with the assigned classification label is added to the updated labeled training dataset 806.

Using the updated labeled training dataset 806, the classification neural network 104 is re-trained. For example, the updated labeled training dataset 806 may be gradually expanded in due course of operation of the classification neural network 104 on the second computing device 108. After such re-, an accuracy training of the classification neural network 104 may be equal to or upwards of 95%, specifically, equal to or upwards of 97%.

FIG. 8B illustrates an example flowchart 810 of a re-training process of the classification neural network 104, according to some example embodiments. FIG. 8B is explained in conjunction with elements of FIG. 8A.

At 812, a probability score for a classification label corresponding to patient bioelectric data for a patient is determined. For example, the probability score is determined based on probability scores generated by the classification neural network 104 during its operation for classifying the patient bioelectric data into one of different classification labels. In an example, the probability score may be in a range of 0 to 1. In other examples, the probability score may be defined in a range of 0 to 100, in percentage, etc.

At 814, a determination is made to check whether the determined probability score is equal to or greater than a probability threshold. For example, the probability threshold may be 0.9, indicating that the classification neural network 104 is very confident for the patient bioelectric data. In such case, if the determined probability score is less than the probability threshold then the method ends.

Alternatively, at 816, when the determined probability score is greater than the probability threshold, classified patient bioelectric data and the classification label is added to the updated labeled training dataset 806. In this manner, a large amount of data may be accumulated in the updated labeled training dataset 806 in due course of operation.

At 818, the classification neural network 104 is re-trained based on the updated labeled training dataset 806 to further improve its accuracy.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

We claim:

1. A system, comprising:
a memory configured to store a classification neural network and computer-executable instructions; and
one or more processors operably connected to the memory, the one or more processors configured to execute the computer-executable instructions to:
receive a first set of simulated bioelectric signals and patient bioelectric signals from a first computing device and a second set of simulated bioelectric signals from a second computing device;
generate a compensation factor for the second computing device based on the first set of simulated bioelectric signals and the second set of simulated bioelectric signals;
generate compensated patient bioelectric signals based on the compensation factor and the patient bioelectric signals; and
train the classification neural network based on the compensated patient bioelectric signals, the second set of simulated bioelectric signals and the compensation factor, wherein the classification neural network is trained to predict a classification label for each of one or more bioelectric signals.

2. The system of claim 1, wherein:
the first computing device is configured to operate with a first antenna array to detect first scattering data relating to the first set of simulated bioelectric signals and the patient bioelectric signals; and
the second computing device configured to operate with a second antenna array to detect second scattering data relating to the second set of simulated bioelectric signals.

3. The system of claim 1, wherein the classification label relates to a health condition associated with the one or more bioelectric signals.

4. The system of claim 1, wherein the one or more processors are further configured to execute the computer-executable instructions to:
generate the compensation factor based on a difference between the first set of simulated bioelectric signals collected by the first computing device and the second set of simulated bioelectric signals collected by the second computing device.

5. The system of claim 1, wherein the classification neural network comprises one or more batch normalization layers, and wherein the one or more processors are further configured to execute the computer-executable instructions to:
train the one or more batch normalization layers to learn one or more scaling parameters and one or more shifting parameters of training data for normalizing the training data of the second computing device, wherein the training data comprises the compensated patient bioelectric signals and the second set of simulated bioelectric signals.

6. The system of claim 5, wherein the one or more processors are further configured to execute the computer-executable instructions to:
deploy the trained classification neural network on the second computing device, wherein the second computing device is configured to collect patient bioelectric data; and
re-train the one or more batch normalization layers to update the one or more scaling parameters and the one or more shifting parameters based on the patient bioelectric data for normalization thereof.

7. The system of claim 6, wherein the one or more processors are further configured to execute the computer-executable instructions to:
classify the patient bioelectric data using the trained classification neural network to associate at least one classification label with the patient bioelectric data; and
cause to display, using a display associated with the second computing device, the classified patient bioelectric data with the corresponding at least one classification label.

8. The system of claim 7, wherein the one or more processors are further configured to execute the computer-executable instructions to:
determine a probability score for the at least one classification label corresponding to the patient bioelectric data;
on determining the probability score to be greater than a predefined probability threshold, add the patient bioelectric data and the at least one classification label to an updated labeled training dataset; and
re-train the classification neural network deployed on the second computing device based on the updated labeled training dataset.

9. The system of claim 1, wherein the classification neural network includes a plurality of one-dimensional (1D) convolutional neural networks (CNNs).

10. A system, comprising:
a memory configured to store a trained classification neural network and computer-executable instructions; and
one or more processors operably connected to the memory, the one or more processors configured to execute the computer-executable instructions to:
receive patient bioelectric data relating to an anatomical part of a patient;
classify the patient bioelectric data using a trained classification neural network to associate at least one classification label with the patient bioelectric data, wherein
the classification neural network is trained based on patient bioelectric signals collected by a first computing device and compensated based on a compensation factor for a second computing device,
the compensation factor is determined based on a first set of simulated bioelectric signals collected by the first computing device and a second set of simulated bioelectric signals collected by the second computing device, and
the classification label indicates one of: a presence, or an absence of a health condition, associated with the anatomical part; and
output the patient bioelectric data with the corresponding at least one classification label.

11. The system of claim 10, wherein the compensation factor is generated based on a difference between the first set of simulated bioelectric signals and the second set of simulated bioelectric signals.

12. The system of claim 10, wherein, to assign the at least one classification label to the patient bioelectric data using the trained classification neural network, the one or more processors are further configured to execute the computer-executable instructions to:
receive, using an input layer of the classification neural network, the patient bioelectric data detected by the second computing device;
perform, using one or more batch normalization layers of the classification neural network, adaptive batch normalization on the patient bioelectric data based on one or more scaling parameters and one or more shifting parameters;
extract, using one or more feature extraction layers of the classification neural network, high-level features from the patient bioelectric data;
predict, using an output layer of the classification neural network, a probability score for one or more classification labels for the patient bioelectric data; and
output, using the output layer, classified patient bioelectric data based on the probability score, the classified bioelectric signal data comprising at least one classification label.

13. The system of claim 12, wherein, to re-train the trained classification neural network, the one or more processors are further configured to execute the computer-executable instructions to:
add the patient bioelectric data with the corresponding at least one classification label to an updated labeled training dataset based on determining the probability score associated with the at least one classification label for the patient bioelectric data to be greater than a predefined probability threshold; and
re-train the classification neural network deployed on the second computing device based on the updated labeled training dataset.

14. The system of claim 10, wherein the patient bioelectric data corresponds to brain waves of the patient, and wherein the classification label indicates one of: a presence of stroke condition, or an absence of stroke condition.

15. A method, comprising:
receiving a first set of simulated bioelectric signals and patient bioelectric signals from a first computing device and a second set of simulated bioelectric signals from a second computing device;
generating a compensation factor for the second computing device based on the first set of simulated bioelectric signals and the second set of simulated bioelectric signals;
generating compensated patient bioelectric signals based on the compensation factor and the patient bioelectric signals; and
training a classification neural network based on the compensated patient bioelectric signals, the second set of simulated bioelectric signals and the compensation factor, wherein the classification neural network is trained to predict a classification label for each of one or more bioelectric signals.

16. The method of claim 15, wherein the classification label relates to a health condition associated with the one or more bioelectric signals.

17. The method of claim 15, further comprising:
generating the compensation factor based on a difference between the first set of simulated bioelectric signals collected by the first computing device and the second set of simulated bioelectric signals collected by the second computing device.

18. The method of claim 15, wherein, to train the classification neural network, the method further comprises:
deploying the trained classification neural network on the second computing device, wherein the second computing device is configured to collect patient bioelectric data;
classifying the patient bioelectric data using the trained classification neural network to associate at least one classification label with the patient bioelectric data; and
cause to displaying, using a display associated with the second computing device, the classified patient bioelectric data with the corresponding at least one classification label.

19. The method of claim 18, wherein, to train the classification neural network, the method further comprises:
determining a probability score for the at least one classification label corresponding to the patient bioelectric data;
on determining the probability score to be greater than a predefined probability threshold, adding the patient bioelectric data to an updated labeled training dataset; and
re-training the deployed classification neural network based on the updated labeled training dataset.

20. A computer programmable product comprising a non-transitory computer readable medium having stored thereon computer executable instructions, which when executed by one or more processors, cause the one or more processors to carry out operations comprising:
receiving a first set of simulated bioelectric signals and patient bioelectric signals from a first computing device and a second set of simulated bioelectric signals from a second computing device;
generating a compensation factor for the second computing device based on the first set of simulated bioelectric signals and the second set of simulated bioelectric signals;
generating compensated patient bioelectric signals based on the compensation factor and the patient bioelectric signals; and
training a classification neural network based on the compensated patient bioelectric signals, the second set of simulated bioelectric signals and the compensation factor, wherein the classification neural network is trained to predict a classification label for each of one or more bioelectric signals.
